(19) United States
(12) Patent Application Publication
(10) Pub. No.: US 2015/0233907 A1
(43) Pub. Date: Aug. 20, 2015

(54) MICROFLUIDIC CHIP FOR MULTI-ANALYTE DETECTION

(71) Applicant: NANOSPEED DIAGNOSTICS INC., Edmonton (CA)

(72) Inventors: Rajan Gupta, Edmonton (CA); Seema Gupta, Edmonton (CA)

(73) Assignee: NANOSPEED DIAGNOSTICS INC., Edmonton, AB (CA)

(21) Appl. No.: 14/425,522

(22) PCT Filed: Sep. 27, 2013
(86) PCT No.: PCT/CA2013/050736

§ 371 Date: Mar. 3, 2015

Related U.S. Application Data
(60) Provisional application No. 61/706,375, filed on Sep. 27, 2012.

(51) Int. Cl. G01N 33/543

(52) U.S. Cl. CPC G01N 33/54313; G01N 33/54373

(57) ABSTRACT

A microfluidic chip and compatible bio-sensor are provided to detect and/or quantify an analyte in a sample fluid, and preferably to simultaneously quantify multiple analyte(s) in a sample fluid volume. A fluid volume containing microbeads is flowed through an elongate planar sample chamber of the microfluidic chip. Microbead traps or barriers in the sample chamber retain the microbeads. The sample fluid volume is Excitation flowed through the sample chamber. A conjugate specific to the analyte is and labeled with a fluorophore is introduced into the sample chamber. In the biosensor, an excitation wavelength is directed parallel to the plane of the sample chamber. Any fluorescent emissions from the sample chamber are detected in a direction substantially orthogonal to the plane of the sample chamber by a photodetector, and correlated to an amount or concentration length of sample chamber of analyte in the sample fluid volume.

### FIELD OF THE INVENTION

The present invention relates to systems and methods using a microfluidic chip that can be used with microbeads to label multiple analytes with fluorophores or other types of labels for conducting immunoassays, and in particular to a biosensor that can be used to detect and quantify the amount of multiple analytes labeled with fluorophores.

### BACKGROUND OF THE INVENTION

To quantitatively analyze lateral flow strips for rapid diagnostics, which otherwise would only provide purely qualitative results, some developers are focusing on readers for lateral flow strips using off-axis or confocal concepts similar to fluorescent microscopy. Both concepts utilize optics to illuminate and excite a small (point-like) volume on the sample strip (i.e. antibody test- and control-lines). Sensitivity in this case is limited to the sensitivity of the lateral flow strips. Although the detection of multiple analytes is possible (by using multiple test lines on the strip), this would most likely involve moving parts and complex optics, as the focal point of the detector optics would have to scan over the strip.

Fluorometers are devices used to measure parameters of fluorescence, such as the intensity and wavelength distribution of a fluorescent emission spectrum. Typically, a fluorometer is fitted with a glass cuvette to hold the sample for analysis. Analytes with corresponding absorption and emission wavelengths may be extracted into a solvent and inserted in the measuring cuvette. Typically, relatively large volumes of analyte are required. The fluorometer will then determine the fluorescence intensity and determine the analyte concentration if an appropriate calibration sample for said sample is also provided. A popular example of such a device is the Picofluor™ by Turner Biosystems Inc. with two optical excitation wavelengths (UV+blue/green) and a claimed detection limit of 1.0-100 ng/mL.

Accordingly, there is a need in the art for systems and methods of conducting assays that can be used to detect and/or quantify multiple analytes in a sample fluid, and which require relatively small volumes of the sample fluid.

### SUMMARY OF THE INVENTION

In general terms, the present invention provides a microfluidic chip, a biosensor, and methods of detecting at least one analyte in a sample fluid volume.

In one aspect, the invention comprises a microfluidic chip for use with an assay using a plurality of microbeads, the microfluidic chip comprising:
(a) two opposed planar sides defining therebetween an elongate planar sample chamber in fluid communication with an inlet and an outlet, wherein the direction from the inlet to the outlet defines a downstream longitudinal direction and a lateral direction perpendicular to the longitudinal direction in the plane of the sample chamber; and
(b) at least one microbead trap within the sample chamber between the inlet and the outlet.
   Preferably, the volume of the sample chamber is about 10 μL or less. In one embodiment, at least a portion of at least one of the planar sides of the sample chamber is transparent to fluorescent excitation and emission wavelengths, and preferably at least a portion of both planar sides are transparent to fluorescent excitation and emission wavelengths. The chip may comprise an internal reflection waveguide to guide light along the length of the sample chamber.

In one embodiment, the chip may comprise a plurality of microbead traps which each comprise at least two micropillars spaced apart by an intra-trap gap smaller than the microbeads to be retained. The microbead traps are laterally spaced apart to define inter-trap gaps that are larger than the microbeads to be retained. The micropillars of each microbead trap may comprise upstream-facing surfaces that converge laterally toward their respective intra-pair gaps, at an acute angle in the downstream longitudinal direction. In one embodiment, each microbead trap comprises three micropillars laterally spaced apart to define gaps smaller than the microbeads to be retained.

In an alternative embodiment, the at least one microbead trap is formed by a plurality of elongate microwalls in side-by-side relation to define or approximate a plurality of microchannels therebetween, wherein the microchannels have widths smaller than the microbeads to be retained. The trap may comprise a boundary formed by a plurality of elongate microwalls in end-to-end relation with each other to define gaps between the ends, wherein the gaps are smaller than the microbeads to be retained. The at least one microbead trap boundary may comprise a curved barrier forming a bulb-shaped receptacle for the microbeads.

In one embodiment, a microfluidic chip comprises a plurality of microbead traps, divided into at least two groups of microbead traps, which may be interspersed in the sample chamber, or may be separated. A first set of microbead traps may be located in a first region of the sample chamber, and a second set of microbead traps may be located in a second region of the sample chamber spatially separated from the first region, and wherein the first set and second set of microbead traps are sized and arranged to selectively retain different sizes of microbeads. In one embodiment, the second region is located downstream longitudinally of the first region, the first set of microbead traps or barriers are sized and arranged to selectively retain larger microbeads, and the second set of microbead traps or barriers are sized and arranged to selectively retain smaller microbeads.

In one embodiment, the microfluidic chip may further comprise a shield that opaquely masks at least one of the planar sides in either the first region or the second region to a selected emission wavelength.

In another aspect, the invention may comprise a method of detecting and/or quantifying at least one analyte in a sample fluid volume, the method comprising the steps of:
(a) providing a microfluidic chip defining an elongate planar sample chamber in fluid communication with an inlet and an outlet, and comprising at least one microbead trap within the sample chamber between the inlet and the outlet;
(b) causing microbeads to be retained by the at least one microbead trap by flowing a fluid containing a plurality of microbeads through the sample chamber, wherein the microbeads each comprise a binding conjugate specific to the at least one analyte;
(c) introducing the sample into the inlet and allowing the sample to flow through the sample chamber to the outlet such that any analyte present in the sample binds to the binding conjugates;
(d) introducing a fluorophore into the sample chamber, wherein the fluorophore is bound to a labelling conjugate specific to the at least one analyte, or is bound to a control analyte which competes with the at least one sample analyte for binding to the binding conjugates;
(e) directing an excitation wavelength into the sample chamber in a direction substantially parallel to the plane of the sample chamber; and
(f) detecting any fluorescent emissions or absence thereof emitted from the sample chamber in a direction substantially orthogonal to the plane of the sample chamber.
   In one embodiment, the method may further comprise the step of measuring an intensity of the emission wavelength and correlating the intensity to an amount or concentration of the analyte in the sample fluid volume.

In one embodiment, the method may be adapted to detect a first analyte and a second analyte, comprising the steps of retaining microbeads specific to both the first analyte and the second analyte in the sample chamber; using a first labelled conjugate specific to the first analyte and a second labelled conjugate specific to the second analyte; and detecting the presence or absence of the first and second labels. The microbeads may comprise a first set of microbeads having a defined size and adapted to bind to the first analyte, and a second set of microbeads having a defined size smaller than the first microbeads and adapted to bind to the second analyte, wherein the microfluidic chip comprises a plurality of first microbead traps each having a size to retain microbeads of the first set but allowing microbeads of the second set to pass through, and a plurality of second microbead traps each having a size to retain microbeads of the second set.

In one embodiment, the first microbead traps may be located in a first region of the sample chamber, and the second set of microbead traps may be located in a second region of the sample chamber spatially separated from the first region.

In one embodiment, the first labeled conjugate comprises a first fluorophore, and the second labelled conjugate comprises a second fluorophore, and the first and second fluorophores may have either different excitation wavelengths, or different emission wavelengths, or both different excitation wavelengths and different emission wavelengths. The first labeled conjugate and the second labeled conjugate may be trapped in separate regions of the sample chamber The first analyte and the second analyte may be detected separately by performing one or a combination of:
(a) if the first and second fluorophores have different excitation wavelengths, directing the different wavelengths into the sample chamber; and
(b) if the first and second fluorophores have different emission wavelengths, detecting the different emission wavelengths.

In another aspect, the invention comprises a biosensor for detecting a fluorophore in an elongate sample chamber defining a longitudinal direction, the biosensor comprising:
(a) an excitation/emission chamber for retaining the sample chamber;
(b) an excitation light source directed at the sample chamber along the longitudinal direction; and
(c) at least one photodetector positioned to detect light emissions from the sample chamber in a direction substantially orthogonally to the longitudinal direction.

In one embodiment, the biosensor comprises two photodetectors, wherein the excitation/emission chamber retains the sample chamber between the two photodetectors.

One of the two photodetectors may comprise a first optical filter that is opaque to a selected emission wavelength, and wherein the other of the two photodetectors is either optically unfiltered or comprises a second optical filter that is transparent to the selected emission wavelength,

In one embodiment, the biosensor comprises a focusing lens that directs light emitted from the excitation light source at the sample chamber along the longitudinal direction. In one embodiment, the biosensor comprises an optical filter positioned between the excitation light source and the excitation/emission chamber. In one embodiment, the biosensor defines an aperture positioned between the excitation/emission chamber and the at least one photodetector, wherein the aperture is oriented to prevent light from the excitation light source reaching the at least one photodetector.

### BRIEF DESCRIPTION OF THE DRAWINGS

In the drawings, like elements are assigned like reference numerals. The drawings are not necessarily to scale, with the emphasis instead placed upon the principles of the present invention. Additionally, each of the embodiments depicted are but one of a number of possible arrangements utilizing the fundamental concepts of the present invention. The drawings are briefly described as follows:

FIGS. 1A and 1B show a schematic of a prior art off-axis and prior art confocal fluorescent readers, respectively.

FIG. 2 shows a schematic representation of the orthogonal excitation and emission directions used in the biosensor of the present invention.

FIG. 3 shows a schematic representation of one embodiment of a biosensor of the present invention that may be used to read microfluidic chips of the present invention.

FIG. 4A shows a schematic representation of spatial separation for multiple analyte detection. FIG. 4B shows a schematic representation of spectral separation for multiple analyte detection.

FIG. 5 shows a simulation of fluid flow contours around a microbead trapped by micropillars in one embodiment of a microfluidic chip of the present invention.

FIG. 6A shows a schematic perspective view of one embodiment of a microfluidic chip of the present invention. FIG. 6B shows a plan view of the microfluidic chip of FIG. 6A. FIG. 6C shows a detail of the sample chamber and micropillars of one embodiment of the microfluidic chip of FIG. 6A. FIG. 6D shows a top view of the microfluidic chip of FIG. 6A. FIG. 6E shows a detail view of the sample chamber and micropillars of the microfluidic chip of FIG. 6A.

FIG. 7A shows a top view of an alternate embodiment of the microfluidic chip of the present invention. FIG. 7B shows a detail view of the sample chamber and micropillars of the microfluidic chip of FIG. 7A. FIG. 7C shows detail views of micropillars from different regions of the microfluidic chip of FIG. 7A.

FIG. 8A shows a further alternate embodiment of the microfluidic chip of the present invention. FIG. 8B shows a detail view of the sample chamber and micropillars of the microfluidic chip of FIG. 8A. FIG. 8C shows detail views of micropillars from different regions of the microfluidic chip of FIG. 8A.

FIG. 9A shows a further alternate embodiment of the microfluidic chip of the present invention. FIG. 9B shows a detail view of the sample chamber and microwalls of the microfluidic chip of FIG. 9A. FIG. 9C shows detail views of the microwalls from different regions of the microfluidic chip of FIG. 9A.

FIG. 10A shows a further alternate embodiment of the microfluidic chip of the present invention. FIG. 10B shows a detail of the sample chamber and microwalls of the microfluidic chip of FIG. 10A.

FIG. 11 shows a further alternate embodiment of the microfluidic chip of the present invention, having a curved microbead barrier formed my micropillars and microwalls.

FIG. 12 shows a further alternate embodiment of the microfluidic chip of the present invention, having a series of capillary micropillars that approximate microchannels.

FIG. 13 shows a further alternate embodiment of the microfluidic chip of the present invention, having micropillars, capillary micropillars, and microwalls.

FIGS. 14A and 14B shows bright field and fluorescence microscopy images respectively of 100 μm sized polystyrene microbeads placed on glass slide.

FIGS. 15A and 15B shows bright field and fluorescence microscopy images respectively of 100 μm sized polystyrene microbeads trapped within one embodiment of a microfluidic chip of the present invention with micropillar traps.

### DETAILED DESCRIPTION OF PREFERRED EMBODIMENTS

The invention relates to a novel microfluidic chip, methods of detecting or quantifying at least one analyte, and a biosensor. When describing the present invention, all terms not defined herein have their common art-recognized meanings. To the extent that the following description is of a specific embodiment or a particular use of the invention, it is intended to be illustrative only, and not limiting of the claimed invention. The following description is intended to cover all alternatives, modifications and equivalents that are included in the spirit and scope of the invention, as defined in the appended claims.

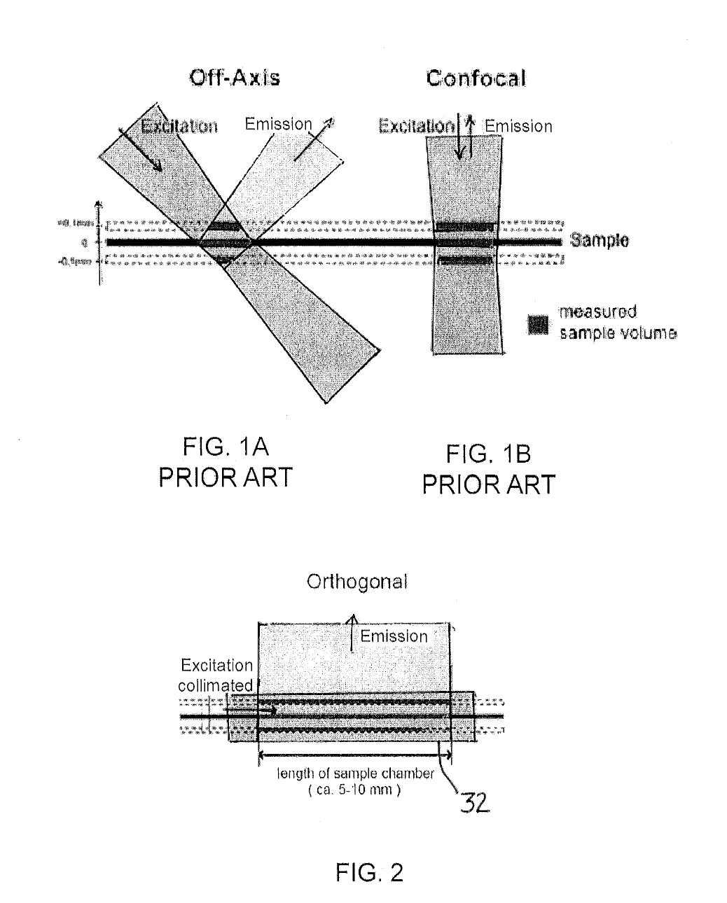

The biosensor (10) and microfluidic chip (30) of the present invention employ a different excitation and emission geometry than those found in the prior art. The concepts of confocal and off-axis illumination as practiced in the prior art, are shown schematically in FIGS. 1A and 1B, respectively. In contrast as shown schematically in FIG. 2, in the biosensor (10) of the present invention, excitation light illuminates the complete length of an elongate planar sample chamber (32) of the microfluidic chip (30). The biosensor (10) has photodetectors (20) that are positioned to detect any light emitted from the sample chamber (32) in a direction substantially orthogonal to the plane of incidence.

The fluorescence intensity of a given amount of a fluorescent label (a fluorophore) depends on (i) its extinction coefficient (i.e. how much excitation light is absorbed), (ii) the path-length in which excitation takes place, and (iii) its quantum yield (i.e. the excitation-to-emission ratio). While the extinction coefficient, quantum yield, and absorption and emission wavelengths are properties of the fluorophore, the design of the biosensor (10) and the microfluidic chip (30) of the present invention aim to optimize excitation of the fluorophore with a long excitation path-length and a large detection area, while using a relatively small volume of sample fluid. The biosensor (10) and microfluidic chip (30) of the present invention are now described in greater detail.

Biosensor

In general, the biosensor (10) of the present invention comprises an excitation/emission chamber (12), an excitation light source (14), and at least one photodetector (16). The biosensor (10) may be used with a microfluidic chip (30) of the present invention which has an elongate sample chamber (32).

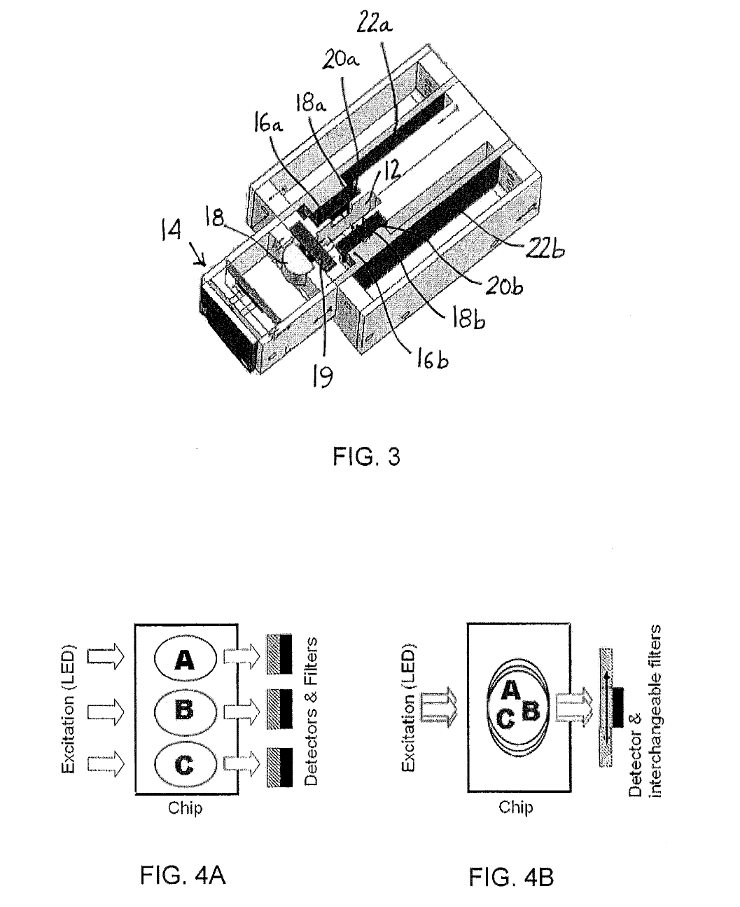

FIG. 3 shows one embodiment of the biosensor (10). The excitation/emission chamber (12) holds the microfluidic chip (30) (not shown). The excitation light source (14) emits excitation light to the microfluidic chip (30). In one embodiment, the excitation light source (14) comprises a light-emitting diode (LED). A focusing lens (18) directs the excitation light through an optical filter (19) at the microfluidic chip (30) along the length of the sample chamber (32). In addition, in one embodiment, a light guiding effect may be achieved with internal reflection waveguide within the microfluidic chip (30), which reduces scattering of the excitation light. Two photodetectors (16a, 16b) are positioned in close proximity to the sample chamber (32) of the microfluidic chip (30). Preferably, the photodetectors (16a, 16b) are placed as close as possible to the microfluidic chip (30), with the sample chamber (32) being positioned between the two photodetectors (16a, 16b), if the microfluidic chip (30) material is transparent on both sides to the emission wavelength, for greater detection sensitivity. The biosensor (10) defines apertures (18a, 18b) between the excitation/emission chamber (12) and the photodetectors (16a, 16b). The apertures (18a, 18b) are oriented to prevent the excitation light from reaching the photodetector and to ensure that only light emitted from the sample chamber (32) reaches the photodetectors (16a, 16b) after passing through optional filters (20a, 20b) which are transparent to the fluorophore emission wavelength, but opaque to the wavelength of the excitation light.

By arranging for the direction of the excitation light to be orthogonal to the detecting surface of the photodetectors (16a, 16b), the light guiding effect of the microfluidic chip (30) ensures that only a small amount of scattered excitation light illuminates the photodetectors (16a, 16b), thus greatly increasing the signal-to-noise ratio of the photodetectors (16a, 16b). Orthogonal incidence of a collimated excitation light beam onto filters (20a, 20b) and microfluidic chip (30) surfaces reduces scattering of excitation light and shifting of filter bands. Therefore less excitation light, which would otherwise be detected by the photodetectors (20a, 20b) as undesired background noise, reaches the photodetectors (20a, 20b). As may be seen, the excitation light, which may in one embodiment be mostly UV or near-UV wavelengths, has a long absorption path, which increases the excited sample amount and thereby the visible fluorescence to be detected. Further, the novel setup of the biosensor (10) allows large area silicon photodetectors (16) to be placed close to the sample on both sides of the sample chamber (32), thereby maximizing the amount and percentage of emitted light that can be detected.

In one embodiment of the biosensor (10) as shown in FIG. 3, the photodetectors (16a, 16b) are photodiodes that are operatively connected to electronic circuit boards (22a, 22b). In one embodiment, the electronic circuits (22a, 22b) may include electronic signal amplifiers used to combine and amplify the signals generated by photodetectors (16a, 16b) located on opposite sides of the sample volume contained by the microfluidic chip (30). Gain switching means may be used to increase the dynamic range of detectable concentrations in the sample fluid, although one skilled in the art will realize that the dynamic range also strongly depends on the specific fluorophore used. In one embodiment, the assembly of the photodetectors (16a, 16b) and the amplifier of the electronic circuits (22a, 22b) are preferably shielded to maintain the highest signal-to-noise ratio. Implementation of excitation source modulation and an integrated lock-in amplifier may be used to significantly increase the signal-to-noise ratio further, thereby lowering the detection limit and extracting low level signals from the photodetectors (16a, 16b). In one embodiment, analog signals generated by the photodetectors (16a, 16b) may be converted to digital signals with an analog-to-digital (A/D) converter integrated with the electronic circuits (22a, 22b). In one embodiment, the data in the signals generated by the photodetectors (16a, 16b) is extracted or transmitted via conventional signal output devices, such as a universal serial bus (USB) port a wireless signal transmitter, or combination thereof.

In one embodiment of the biosensor (10), the biosensor also has a power source for the electronic components of the biosensor (10). The power source may comprise a battery, a USB input, a wall adapter, or a combination of the foregoing. In embodiments where the power source combines a battery and a USB input or a wall adapter, unused power from either the wall adapter or the USB input can be used to charge the battery.

Microfluidic Chip

The microfluidic chip (30) of the present invention comprises microbead traps within a sample chamber (32) to control the flow of microbeads (B). Microbeads (B) and methods for coating or attaching conjugates such as antibodies to the microbeads are well known in the art and commercially available.

In one embodiment, the microfluidic chip (30) comprises two opposed planar sides that define between them a planar, elongate sample chamber (32). The sample chamber (32) is in fluid communication with an inlet (34) and an outlet (36). In between the inlet (34) and the outlet (36), the sample chamber (32) has a plurality of internal microbead traps or barriers that may be formed by a plurality of micropillars (40) or microwalls (50), or the side wall barriers (39) of the sample chamber (32), or a combination of these features. In addition, elongate capillary micropillars (40) and microwalls (50) may form or approximate microchannels (52) that enhance the capillary effect of the fluid flow through the sample chamber (32) from the inlet (34) to the outlet (36).

As used herein, the term “longitudinal” in reference to the microfluidic chip (30) shall mean the direction in the plane of the sample chamber (32) defined between the inlet (34) and the outlet (36). As used herein, the term “upstream” when used to describe the position of a first element relative to a second element of the microfluidic chip (30) shall mean that the first element is longitudinally more proximal to the inlet (34) than is the second element. As used herein, the term “downstream” to describe the position of a first element relative to a second element of the microfluidic chip (30) shall mean that the first element is longitudinally more proximal to the outlet (36) than is the second element. As used herein, the term “lateral” in reference to the microfluidic chip (30) shall mean the direction in the plane of the sample chamber (32) that is perpendicular to the “longitudinal” direction.

In one embodiment for use with fluorometric assays, one end wall of the microfluidic chip, or a portion of one end wall, is transparent to an excitation light frequency, allowing the excitation light to longitudinally enter the sample chamber. The micropillars and microwalls (40) within the sample chamber are preferably transparent to either or both the excitation and emission wavelengths as well.

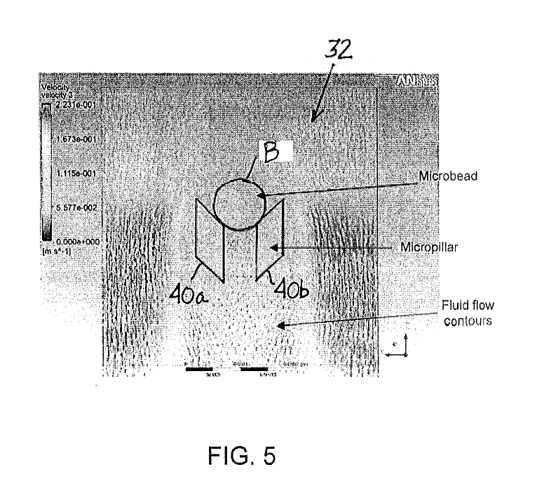

FIG. 5 shows a flow simulation through a sample chamber (32) of one embodiment of the invention with fluid flow streak lines around microbead (B) which is trapped by a pair of micropillars (40a, 40b). The geometrical shape of the micropillars (40) and the spacing between micropillars (40) are designed such that the microbead (B) is trapped upstream of the micropillars (40a, 40b) as the sample fluid flows longitudinally through the sample chamber (32) from the inlet (34) to the outlet (36).

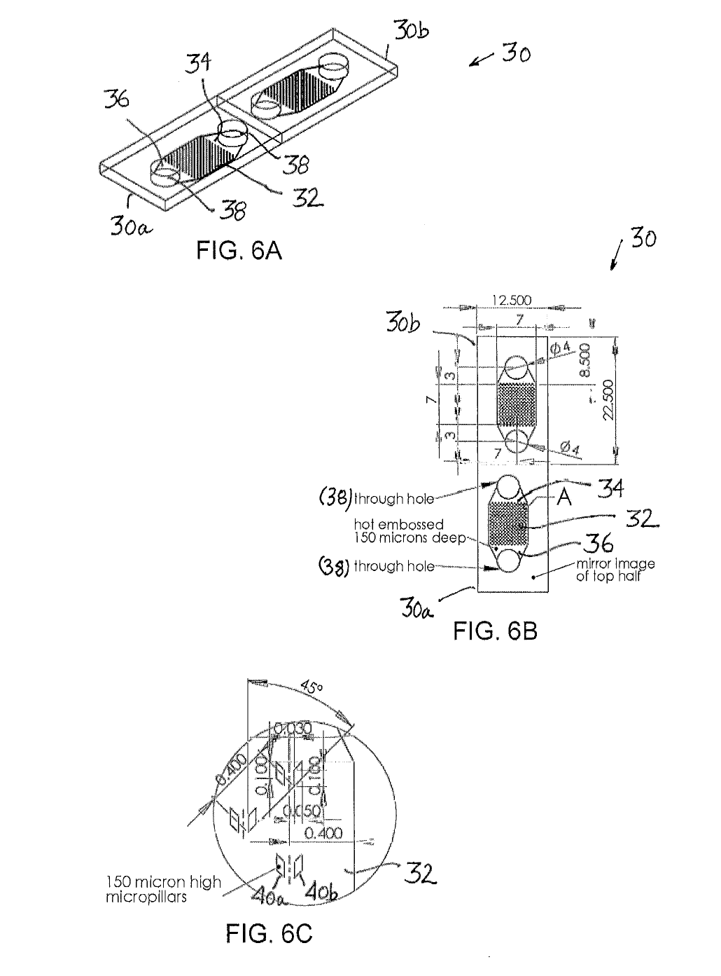

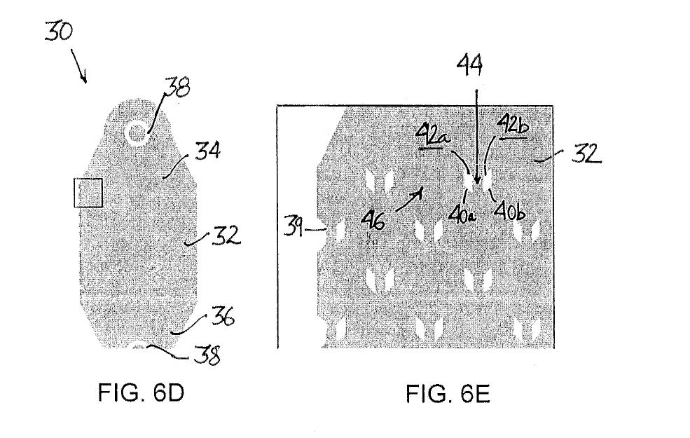

FIGS. 6A through 6E show one embodiment of the microfluidic chip (30). The dimensions shown in FIG. 6A, 6B, and 6C, expressed in millimeters unless otherwise expressed in microns, are exemplary only and not limiting of the present invention. The microfluidic chip (30) comprises two substantially planar sides (30a, 30b), which are mirror images of each other. When the two sides (30a, 30b) are fitted together, they define the sample chamber (32) between them. The two sides (30a, 30b) of the microfluidic chip (30) may be bonded using conventional bonding methods, such as thermal bonding, chemical bonding, or combinations of thermal and chemical bonding. At least one of the two sides (30a, 30b) is transparent to excitation and emission wavelengths. In a preferred embodiment, both of the two sides (30a, 30b) are transparent to the emission wavelengths. An inlet (34) and an outlet (36) provide fluid communication with through holes (38) and the sample chamber (32). The through holes (38) create reservoirs that can receive a deposit of fluid. The sample chamber (32) comprises a plurality of internal micropillars (40a, 40b) in between the inlet (34) and the outlet (38).

FIGS. 6D and 6E show one embodiment of the micropillars (40) in which they have a cross-sectional shape of a parallelogram. The micropillars (40) are arranged in micropillar pairs in which the micropillars (40a, 40b) are laterally separated to define a small intra-pair gap (44) between them. The upstream face (42a) of the micropillar (40a) and the upstream face (42b) of the other micropillar (40b) form an acute angle between that converges towards the intra-pair gap (44) in the direction from the inlet (34) to the outlet (36). The intra-pair gap (44) is smaller than the microbead (B) size used in an assay, but the size of the lateral inter-pair gap (46) between different micropillar pairs (40a, 40b) is larger than the microbead (B) size. Accordingly, as a fluid containing the microbeads (B) flows through the sample chamber (32) from the inlet (34) to the outlet (44), the microbeads (B) become seated on the leading faces (42a, 42b) and are thereby retained by the micropillar pairs (40a, 40b) throughout the sample chamber (32). As shown in FIG. 6E, the outside column of micropillars (40) coordinates with contours of side wall barriers (39) of the sample chamber (32) to form microbead traps along the periphery of the sample volume. The lateral gaps between the micropillars (40) and the side wall barriers (39) are smaller than the microbead (B) size.

Each pair of micropillars may be sized to retain a single microbead, in which case it may be seen that trapped microbeads will be relatively uniformly dispersed through the sample chamber if sufficient microbeads are used.

It is preferred that the micropillars (40) are arranged to trap a substantial majority of the microbeads (B) present in the sample, which will result in increased signal strength. Spacing of the micropillars (40) may be varied, and the orientation of the micropillars may also be varied, with some or all of the micropillars oriented along the fluid flow path, or at an angle or orthogonal to fluid flow.

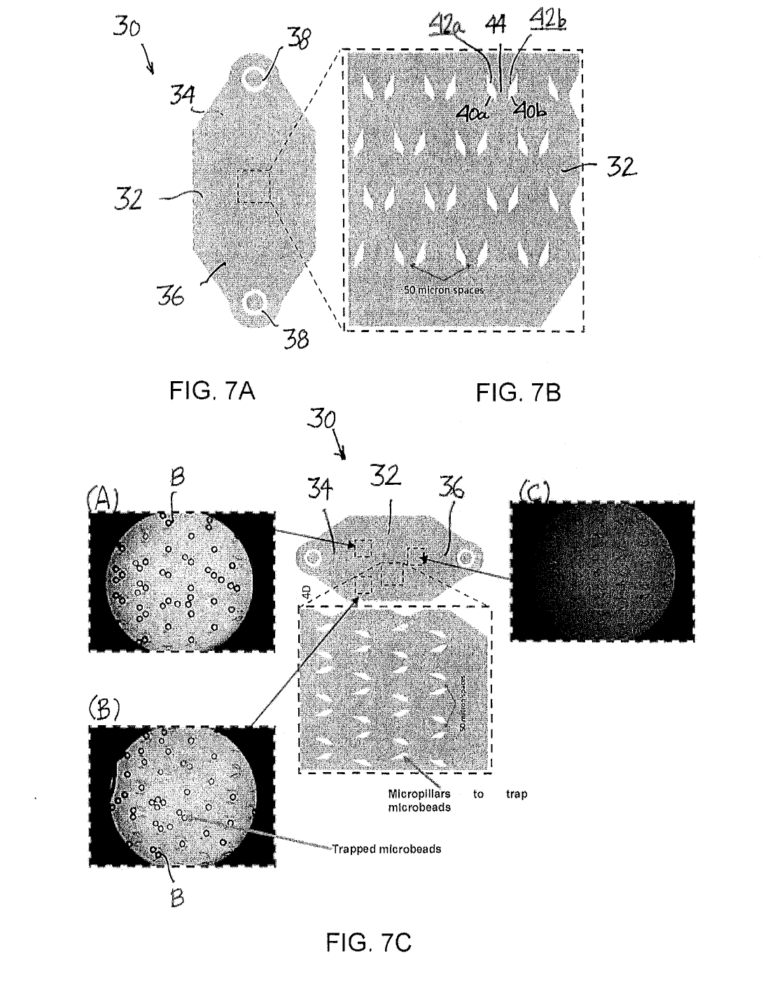

FIGS. 7A and 7B show an alternate embodiment of the microfluidic chip (30) where the micropillars (40) have a cross-sectional shape of an irregular quadrilateral. Also, the upstream faces (42a, 42b) of the pair of micropillars (40a, 40b) converge at a more acute angle than in the embodiment shown in FIG. 6E. This may result in more effective trapping of a microbead (B). FIG. 7C shows microbead (B) trapping in the micropillar (40) design and arrangement of FIG. 7B. With this design, it is seen that more microbeads (B) are trapped in the upstream regions (A, B) closer to the inlet (34), while no microbeads (B) are shown in the downstream region (C) closer to the outlet (36).

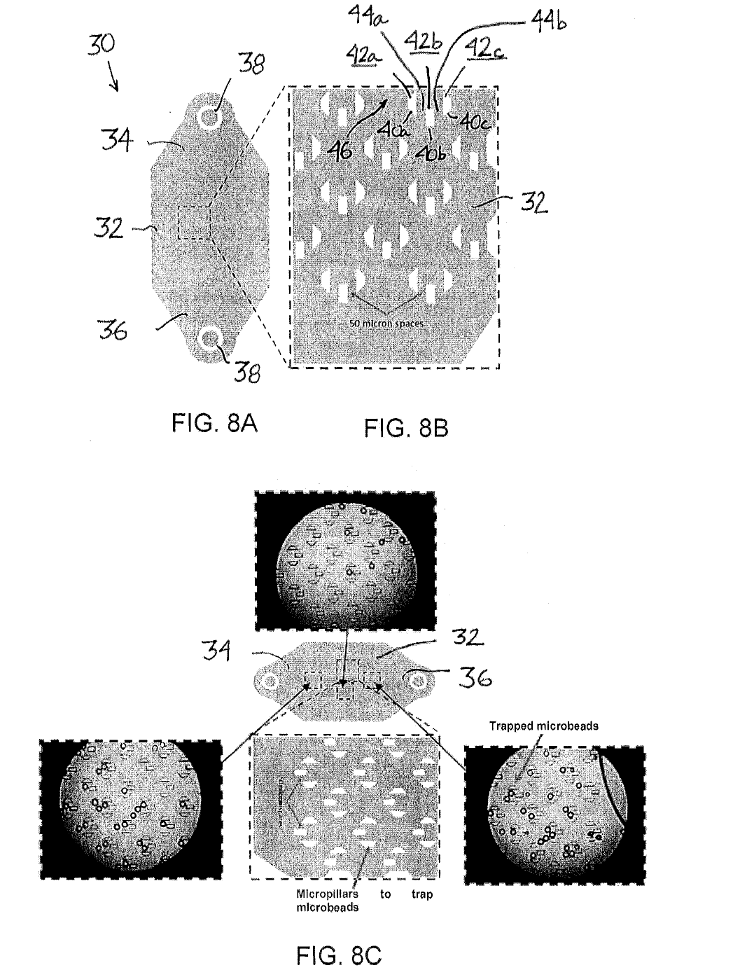

FIGS. 8A through 8C show an alternative arrangement of the micropillars (40). The micropillars (40) are arranged in micropillar triplets (40a, 40b, 40c). Two micropillars (40a, 40c) have a cross-sectional shape of a parallelogram. The third micropillar (40b) has the cross-sectional shape of a rectangle, and is positioned laterally between the micropillars (40a, 40c). The upstream face (42b) of the micropillar (40b) is positioned within the longitudinal span of the micropillars (40a, 40c). The upstream faces (42a, 42c) of the micropillars (40a, 40c) diverge from the intra-triplet gaps (44a, 44b) in the downstream longitudinal direction. The lateral intra-triplet gaps (44a, 44b) are smaller than the microbead (B) size used in an immunoassay, but the lateral inter-triplet gaps (46) between the different micropillar triplets (40a, 40b, 40c) are larger than the microbead (B) size. Accordingly, as a fluid containing the microbeads (B) flows through the sample chamber (32) from the inlet (34) to the outlet (36), the microbeads (B) become seated between the micropillars (40a, 40c) and seated on the upstream faces (42b) and are thereby trapped by the micropillar triplets (40a, 40b, 40c) throughout the sample chamber (32). This arrangement of micropillars (40) facilitates the flow of microbeads (B) and fluid around a micropillar triplet (40a, 40b, 40c) which has already trapped a microbead. Photomicrographs of microbead trapping in different areas of the sample volume are shown in FIG. 8C.

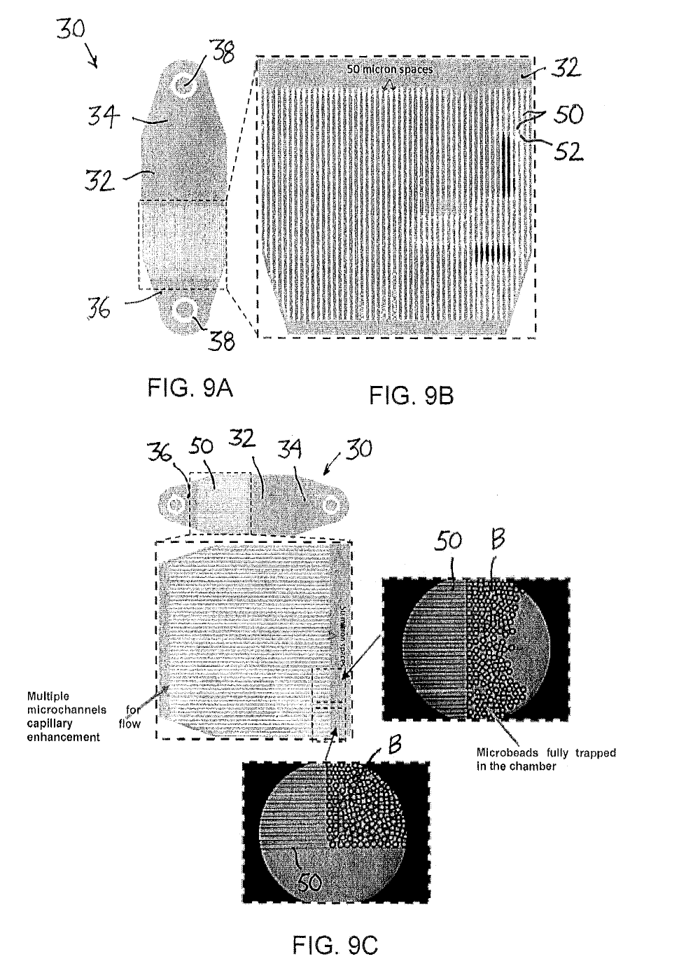

FIG. 9A and 9B show an alternative embodiment of the microfluidic chip (30). A portion of the sample chamber (32) has a plurality of elongate microwalls (50) laterally spaced apart in side-by-side parallel relation to each other to define a plurality of microchannels (52) between them which lead to the outlet (36). The width of the microchannels (52) is selected such that microbeads (B) will not enter the microchannels (52). Microchannels (52) increase the capillary effect and may enhance flow of reagents from the inlet (34) to the outlet (36). Microchannels (52) may also reduce the possibility of an air bubble being trapped within the sample chamber (32). Photomicrographs of microbeads (B) being trapped in front of the microchannels (52) are shown in FIG. 9B.

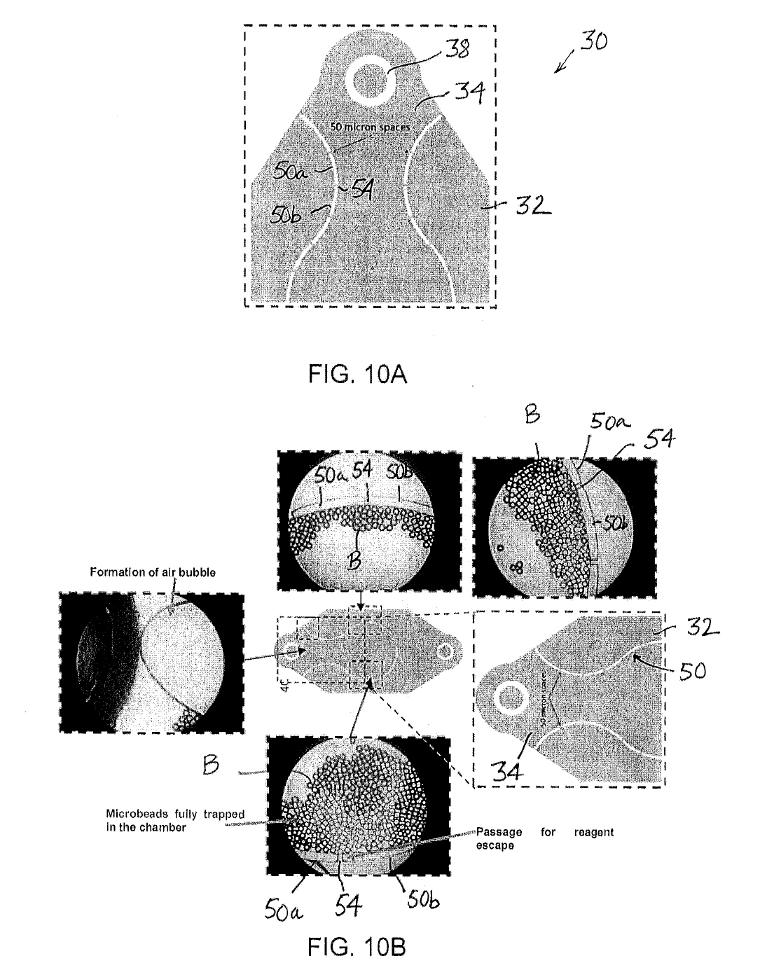

FIGS. 10A and 10B show an alternative embodiment of the microfluidic chip (30) having a microbead trap in the form of a barrier between the inlet (34) and the outlet (36). The microbead barrier is made of up of a plurality of microwalls (50a, 50b) placed in end-to-end relationship with each other. The barrier is curved around the inlet (34) to define a bulb-shaped receptacle for the microbeads (B) between the inlet (34) and the outlet (44) and having a narrowing section proximal to the inlet (34). Gaps (54) between the ends of the microwalls (50a, 50b) are selected to have sizes which permit fluid flow through the barrier, but are smaller than the microbead (B) size to prevent passage of microbeads (B). Accordingly, the microbeads (B) will accumulate upstream of the barrier on the side proximal to the inlet (34). The microbead barrier may be combined with microbead traps formed from micropillars (40) and other microwalls (50) that form microchannels (52).

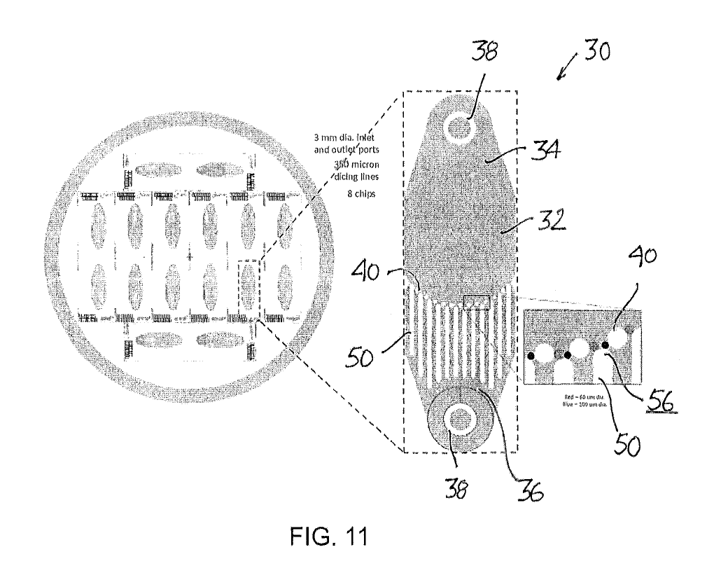

FIG. 11 shows yet another alternative embodiment of the microfluidic chip (30). A portion of the sample chamber (32) has a plurality of elongate microwalls (50) laterally spaced apart in parallel relation to each other to define a plurality of microchannels (52) between them which lead to the outlet (36). The microwalls (50) have rounded upstream ends (56). Micropillars (40) having a round cross-sectional shape are positioned longitudinally upstream of and laterally between the upstream ends (56), in close proximity to the upstream ends (56). The upstream ends (56) of the microwalls (50) and the micropillars (40) define gaps therebetween that are smaller than the microbead (B) size to prevent passage of microbeads (B). As such, the micropillars (40) and the microwalls (50) cooperate to form a curved microbead barrier.

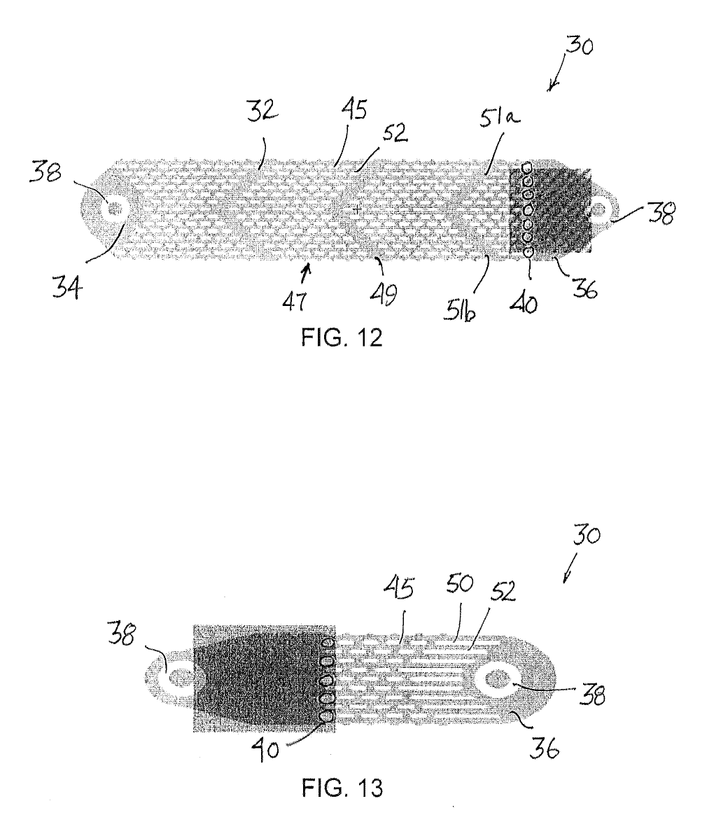

FIG. 12 shows an alternative embodiment of the microfluidic chip (30). A series of elongate capillary micropillars (45) approximate microchannels (52) between them. The series of capillary micropillars (45) are grouped in chevron-shaped groupings (47) separated by V-shaped gaps (49) having arms (51a, 51b) that diverge laterally in the downstream longitudinal direction. Within each grouping, laterally adjacent capillary pillars (40) are longitudinally staggered in respect to each other. The capillary micropillars (40) promote uniform fluid flow in the sample chamber (32) and along the capillary micropillars (40), and may minimize air traps or voids in the sample chamber (32). In one embodiment, small micropillars (40) having widths equivalent to the widths of 2 to 3 capillary micropillars (45) may be formed near the outlet (36). The microbeads (B) are trapped along a barrier formed at the entrance to the capillary pillars (40) in the shaded square.

FIG. 13 shows an alternative embodiment of the microfluidic chip (30). The capillary micropillars (45) are combined with elongate microwalls (50) that define microchannels (52). Capillary micropillars (40) appear to lessen the chance of air bubble traps. Small micropillars (40) having widths equivalent to the widths of 2 to 3 capillary micropillars (40) are formed sometimes near the inlet (34). The microbeads (B) are trapped along a barrier formed at the entrance to the capillary pillars (40) in the shaded square.

Use and Operation of the Microfluidic Chip and Biosensor

Exemplary use and operation of the microfluidic chip (30) of the present invention is now described in one embodiment. As used herein, the term “conjugate” refers to a chemical that is capable of specifically interacting with an analyte to form an analyte-conjugate complex, and includes, without limitation, an antibody. As used herein, the term “antibody” refers to an immunological protein that is capable of specifically binding with a specific antigen that is part of the analyte, and includes a fragment of a protein that exhibits such functionality.

In general terms, embodiments of the microfluidic chip and biosensor may be used for any suitable assay, such as fluorescence-based assays, including intensity-measurement based assays, and fluorescence resonance energy transfer assays. In one embodiment, the assay comprises immunoassays to detect and/or quantify analytes in a sample.

In an exemplary operation of one embodiment of the microfluidic chip (30), microbeads (B) of an appropriate diameter are coated with antibody specific to the analyte of interest and dispersed into a fluid volume. Microbeads (B) which are suitable for coating with antibodies are well known in the art and commercially available. The fluid volume is deposited onto the microfluidic chip (30) near the inlet (34). As the first fluid volume is drawn through the sample chamber (32) towards the outlet (36), the microbeads (B) are trapped in the sample chamber (32) by the microbead traps or microbead barriers formed by the micropillars (40) or microwalls (50), or a combination of them.

Next, a sample fluid volume that is to be tested for the analyte of interest is deposited onto the microfluidic chip (30) in the through hole (38) near the inlet (34). As the sample fluid volume is drawn through the sample chamber (32) toward the outlet (36), any analyte present in the sample volume is bound to the antibody coated on the microbeads (B) retained in the sample chamber (32).

Next, a fluid volume containing labeled antibodies that are specific to the analyte is deposited onto the microfluidic chip (30) near the inlet (34). As the sample fluid volume is drawn through the sample chamber (32) toward the outlet (36), the labeled antibodies bind to any analyte that may be bound to the antibodies coated onto the trapped microbeads (B).

In one embodiment, the label may be a fluorophore for a fluorescence-based immunoassay. Fluorescently labeled antibodies are well known in the art. Immunoassays using detection labels other than a fluorophore may be used. Non-limiting examples of such labels include a radiolabel, a chromogen, catalyst, fluorescent compound, chemiluminescent compound, colloidal gold, a dye particle, a latex particle tagged with a detector reagent such as, for example, a colored or fluorescent dye, and the like.

In other embodiments, other immunoassay protocols may be suitable, including competitive immunoassays. By way of a non-limiting example, a control sample fluid volume containing a labeled analyte, and a test sample fluid volume suspected to contain unlabelled analyte may be introduced into the sample chamber. The labeled analyte in the control volume and any of the unlabelled analyte in the test volume compete against each other to bind to antibodies coated onto the microbeads. The more unlabelled analyte that is present in the test sample fluid volume, the more that the labeled analyte is competed against binding to the antibody coated onto the microbeads (B). Thus, the amount of fluorophore in the sample chamber may be negatively correlated to the amount of analyte in the test sample fluid volume.

In other embodiments, non-antibody conjugates may be used in other types of analyte detection assays. As non-limiting examples, such conjugates may include biotin and avidin or streptavidin, and modified forms thereof. By way of non-limiting example, a sample fluid volume suspected to contain an analyte may be biotinylated with a fluorophore-labeled biotin, and introduced into the sample chamber containing microbeads coupled with avidin or streptavidin. If the biotinylated analyte is present in the sample fluid volume, the fluorophore-labeled biotin will bond with the avidin or streptavidin coupled to the microbeads.

In order to detect and quantify the amount of analyte within the sample fluid, the biosensor (10) may be used with the microfluidic chip (30). The microfluidic chip (30) is placed in the excitation/emission chamber of the biosensor (10). The excitation light source (14) is used to excite the antibodies labeled with fluorophore within the sample chamber (32), thereby causing them to emit light. The emitted light is detected by the photodetectors (20) which in turn generated electronic signals. The electronic signals may be processed to determine signal strength, which may be correlated to an amount or concentration of labeled antibody present in the sample chamber (32), which may in turn be positively or negatively correlated to an amount or concentration of analyte in the sample chamber (32).

In one embodiment, the biosensor (10) and the microfluidic chip (30) enable the detection of multiple analytes in a single sample of biological fluid without having to rely on any moving parts within the biosensor (10). It is often necessary or desirable to determine the presence and/or concentration of multiple analyte targets within a single biological fluid sample (e.g. blood) to accurately diagnose a medical condition or disease state. As the concentration of each analyte has to be detected independently from the others, the fluorescence signals emitted by each analyte must be separated either spatially or spectrally (by the wavelength of either absorbed or emitted light), or both. Spatial and spectral separation strategies are shown conceptually in FIGS. 4A and 4B, respectively.

In one embodiment, multiple analyte detection may be based on spectral separation strategies. For example, a first subset of fluorescently labeled antibodies is specific to a first analyte, while a second subset of fluorescently labeled antibodies is specific to a second analyte. The first and second types of fluorescently labelled antibodies are labelled with different fluorophores which fluoresce at different wavelengths. Detection of the different wavelength emissions may then permit quantification of the amount or concentration of the two different analytes. In one embodiment, fluorescence signals are separated by choosing either an excitation or emission wavelength (e.g. via optical filters (22a, 22b) where only one analyte can be detected by the photodetector (20a, 20b). Appropriate optical filters may be chosen for each fluorophore used to optimize the signal-to-noise ratio, and make multiple analytes measurable simultaneously without substantial cross-talk.

In an alternative embodiment, multiple analyte detection may be based on spatial separation strategies within the microfluidic chip (30). For example, a first subset of antibody-coated microbeads may have a first diameter, such as 50 μm, and a second subset of antibody-coated microbeads may have a second diameter, such as 100 μm. The first and second subsets are coated with different antibodies specific to different analytes. The microfluidic chip (30) is configured with a first set of microbead traps or barriers which trap the larger microbeads in one region, while the smaller subset passes through, and a second set of microbead traps or barrier which trap the smaller microbeads in a separate region. The sample chamber (32) may have a shield or shields which mask different regions of the microfluidic chip (30) from the photodetectors (16a, 16b), thereby permitting separate detection of the spatially separated fluorescent sources. If spatial separation is achieved on the microfluidic chip (30), the same fluorophore may be used to independently measure the concentrations of all analytes. In this case, however, all fluorophores will emit at the same wavelength. Therefore, a separate photodetector (20) with a filter (22) is required for each target analyte, but a single elongated excitation source is sufficient.

Both spectral and spatial separation strategies may be used in combination, thereby greatly improving the ability to distinguish between the fluorescence signals and increasing the signal-to-noise ratio. In this way the sensitivity of the biosensor (10) can be significantly improved and detection limits lowered.

As will be apparent to those skilled in the art, various modifications, adaptations and variations of the foregoing specific disclosure can be made without departing from the scope of the invention claimed herein.

### EXAMPLES

The following examples are provided to exemplify embodiments of the invention, and are not intended to limit the claimed invention unless explicitly recited in a limiting manner.

### Example 1

### Detection of Different Fluorophores

Experiments using the biosensor (10) to detect different fluorophores (Vitamin A, FITC (fluorescein isothiocyanate), Alexa F1uor™ 430) and according excitation wavelength show good results for sensitivity, dynamic range, linearity, and detection limits. When measuring the auto-fluorescence of Vitamin A in various concentrations, a detection limit of about 1.5 ng/mL was measured. Concentrations as high as 1.5 μg/mL were detected and compared to standard values determined via optical density measurements, resulting in a highly linear curve (R-squared=0.995) over the full dynamic range. Similar results (LOD<50 ng/mL, R-squared>0.99) have been obtained measuring dilutions of FITC and Alexa Fluor™ 430 fluorophores using very small sample volumes (˜10 μL).

### Example 2

### Pre-Coating of Microbeads, Immobilization Protocol and Detection in Microscale

Monoclonal antibodies were used to pre-coat microbeads (B). Chemical linkers were used to immobilize specific monoclonal antibodies to the surface of microbeads (B). The excess of chemical linker and free antibody was removed either by dialysis or using specific columns and buffers. Microbeads (B) coated with the monoclonal antibody in a volume of up to 25 μL were introduced into the microfluidic chip (30) and trapped by the microbead traps. A sample fluid containing the analyte (specific to the monoclonal antibody) was introduced into the microfluidic chip (30). Analytes present in the sample will bind to the monoclonal antibody coated onto the microbeads (B). Following a wash step, a second antibody conjugated to a fluorescence probe was introduced into the microfluidic chip (30) to bind to the analyte(s) already bound onto the trapped microbeads (B). The entire set of immobilization experiments consisted of optimizing the reagent concentration, incubation time and temperatures. The same set of experiments was performed for multiple analytes simultaneously corresponding to the specific monoclonal antibodies. Biological assay corresponds to sandwich ELISA and can be replaced with competitive ELISA for the detection and analysis of analytes.

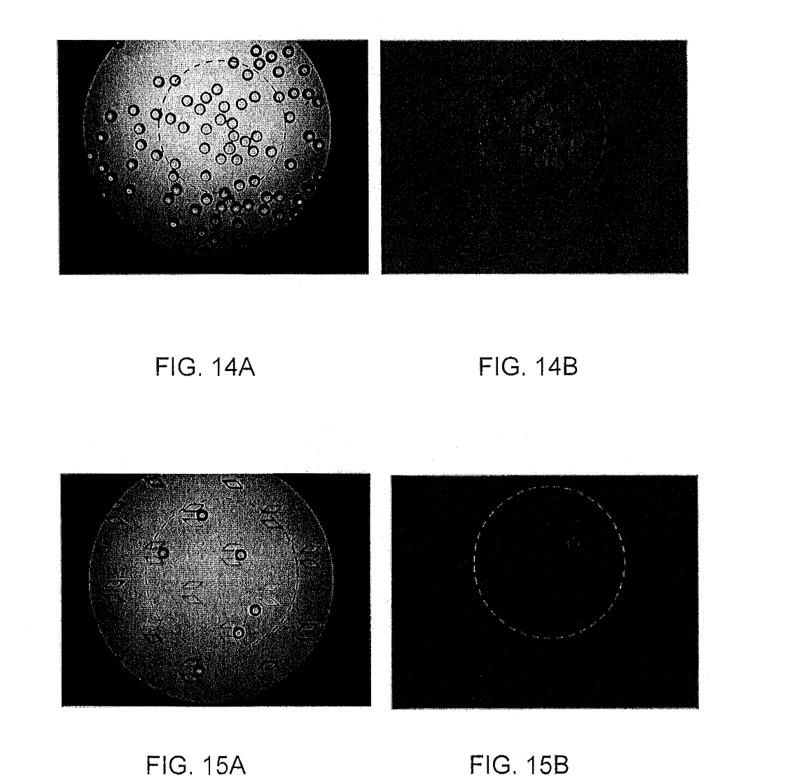

FIGS. 14A and 14B show bright field and fluorescence images respectively of 100 nm sized polystyrene microbeads placed on glass slide. Example includes TSH analytes followed by detection antibodies were immobilized as per the defined immobilization protocol.

FIGS. 15A and 15B show bright field and fluorescence images respectively of 100 nm sized polystyrene microbeads trapped within one embodiment of a microfluidic chip (30) with micropillar traps.

### Example 3

### Microfluidic Chip Biosensor Protocol

Specific antibody coated spherical polymer microbeads (B) are introduced into the microfluidic chip (30), A drop of fresh blood is applied at the inlet (34) of the microfluidic chip (30), The microfluidic chip (30) is incubated at room temperature for 2 to 5 minutes. A wash buffer is introduced into the reaction chamber which replaces the blood sample. A second specific antibody conjugated with a fluorophore is introduced into the sample chamber (32) of the microfluidic chip (30), and is incubated at room temperature for 2 to 5 minutes. Wash buffer is introduced into the reaction chamber which replaces the second conjugated antibody. The fluorescence associated with microbeads (B) is read by inserting the microfluidic chip into the biosensor (10) which is fitted with appropriate filters (22) for the fluorophore. The volume of fluid at each step is preferably about 10 μL or less,

1. A microfluidic chip for use with an assay using a plurality of microbeads, the microfluidic chip comprising:
(a) two opposed planar sides defining therebetween an elongate planar sample chamber in fluid communication with an inlet and an outlet, wherein the direction from the inlet to the outlet defines a downstream longitudinal direction and a lateral direction perpendicular to the longitudinal direction in the plane of the sample chamber; and
(b) at least one microbead trap within the sample chamber between the inlet and the outlet.

2. The microfluidic chip of claim 1 wherein the volume of the sample chamber is about 10 μL or less.

3. The microfluidic chip of claim 1 wherein at least a portion of one of at least one of the planar sides of the sample chamber is transparent to fluorescent excitation and emission wavelengths.

4. The microfluidic chip of claim 3 wherein at least a portion of both planar sides are transparent to fluorescent excitation and emission wavelengths.

5. The microfluidic chip of claim 3 wherein the chip defines an internal reflection waveguide to guide light along the length of the sample chamber.

6. The microfluidic chip of claim 1 comprising a plurality of microbead traps which comprise at least two micropillars spaced apart by an intra-trap gap smaller than the microbeads to be retained.

7. The microfluidic chip of claim 6 wherein the micropillars of each microbead trap comprise upstream-facing surfaces that converge laterally toward their respective intra-pair gaps, at an acute angle in the downstream longitudinal direction.

8. The microfluidic chip of claim 6 wherein each microbead trap comprises three micropillars laterally spaced apart to define gaps smaller than the microbeads to be retained.

9. (canceled)

10. The microfluidic chip of claim 1 wherein the at least one microbead trap is formed by a plurality of elongate microwalls in side-by-side relation to define or approximate a plurality of microchannels therebetween, wherein the microchannels have widths smaller than the microbeads to be retained.

11. The microfluidic chip of claim 1 wherein the at least one microbead trap comprises a boundary formed by a plurality of elongate microwalls in end-to-end relation with each other to define gaps between the ends, wherein the gaps are smaller than the microbeads to be retained.

12. The microfluidic chip of claim 11 wherein the at least one microbead trap boundary comprises a curved barrier forming a bulb-shaped receptacle for the microbeads.

13. The microfluidic chip of claim 1 comprising a plurality of microbead traps, divided into at least two physically separated groups of microbead traps.

14. The microfluidic chip of claim 13 comprising a first set of microbead traps located in a first region of the sample chamber, and a second set of microbead traps located in a second region of the sample chamber spatially separated from the first region, and wherein the first set and second set of microbead traps are sized and arranged to selectively retain different sizes of microbeads.

15. The microfluidic chip of claim 14 wherein the second region is located downstream longitudinally of the first region, the first set of microbead traps or barriers are sized and arranged to selectively retain larger microbeads, and the second set of microbead traps or barriers are sized and arranged to selectively retain smaller microbeads.

16. The microfluidic chip of claim 14 further comprising a shield or filter that opaquely masks at least one of the planar sides in either the first region or the second region to a selected emission wavelength.

17. A method of detecting and/or quantifying at least one analyte in a fluid sample, the method comprising the steps of:
(a) providing a microfluidic chip defining an elongate planar sample chamber in fluid communication with an inlet and an outlet, and comprising at least one microbead trap within the sample chamber between the inlet and the outlet;
(b) causing microbeads to be retained by the at least one microbead trap by flowing a fluid containing a plurality of microbeads through the sample chamber, wherein the microbeads each comprise a binding conjugate specific to the at least one analyte;
(c) introducing the sample into the inlet and allowing the sample to flow through the sample chamber to the outlet such that any analyte present in the sample binds to the binding conjugates;
(d) introducing a fluorophore into the sample chamber, wherein the fluorophore is bound to a labelling conjugate specific to the at least one analyte, or is bound to a control analyte which competes with the at least one sample analyte for binding to the binding conjugates;
(e) directing an excitation wavelength into the sample chamber in a direction substantially parallel to the plane of the sample chamber; and
(f) detecting any fluorescent emissions or absence thereof emitted from the sample chamber in a direction substantially orthogonal to the plane of the sample chamber.

18. The method of claim 17 further comprising the step of measuring an intensity of the emission wavelength and correlating the intensity to an amount or concentration of the analyte in the sample fluid volume.

19. The method of claim 17 adapted to detect a first analyte and a second analyte, comprising the steps of retaining microbeads specific to both the first analyte and the second analyte in the sample chamber; using a first labelled conjugate specific to the first analyte and a second labelled conjugate specific to the second analyte; and detecting the presence or absence of the first and second labels.

20. The method of claim 17 adapted to detect a first analyte and a second analyte, comprising the steps of retaining microbeads specific to both the first analyte and the second analyte in the sample chamber; using a first labelled analyte to compete with a sample first analyte for microbead binding sites, and a second labelled analyte to compete with a second sample analyte for microbead binding sites, and detecting the presence, absence or intensity of any fluorescent emissions.

21-25. (canceled)

26. A biosensor for detecting a fluorophore in an elongate sample chamber defining a longitudinal direction, the biosensor comprising:
(a) an excitation/emission chamber for retaining the sample chamber;
(b) an excitation light source directed at the sample chamber along the longitudinal direction; and
(c) at least one photodetector positioned to detect light emissions from the sample chamber in a direction substantially orthogonally to the longitudinal direction.

27-32. (canceled)
